(19) United States
(12) Patent Application Publication
(10) Pub. No.: US 2024/0058650 A1
(43) Pub. Date: Feb. 22, 2024

(54) ADAPTIVE WORKOUT PLAN CREATION AND PERSONALIZED FITNESS COACHING BASED ON BIOSIGNALS

(71) Applicant: Apple Inc., Cupertino, CA (US)

(72) Inventors: Matthias R. Hohmann, Mountain View, CA (US); Andrea Eppy, Saint Louis, MO (US); Erdrin Azemi, San Mateo, CA (US)

(73) Assignee: Apple Inc., Cupertino, CA (US)

(21) Appl. No.: 18/451,798

(22) Filed: Aug. 17, 2023

Related U.S. Application Data
(60) Provisional application No. 63/373,008, filed on Aug. 19, 2022.

(51) Int. Cl. A63B 24/00; A63B 71/06

(52) U.S. Cl. CPC A63B 24/0062; A63B 71/0686; A63B 2024/0065; A63B 2024/0068; A63B 2071/0625

(57) ABSTRACT

Methods, systems and/or computer-implemented instructions are configured to perform or support actions that include: determining a time contribution for each of a set of workout effort zones for a user, wherein each of the set of workout effort zones corresponds to a range of values for a biosignal; determining a timeseries of workout target effort zones for the user based on the target time contributions for the set of workout effort zones; receiving, during a workout time period, real-time biosignal data from a sensor in a wearable electronic device being worn by the user; generating, during the workout time period, an audio, visual, or haptic stimulus based on the real-time biosignal data and a target effort zone in the time series of workout target effort zones; and outputting, during the workout time period, the audio, visual, or haptic stimulus.

### CROSS-REFERENCE TO RELATED APPLICATIONS

This application is claims priority under 35 U.S.C. § 119(e) to U.S. Provisional Application No. 63/373,008, filed on Aug. 19, 2022, the contents of which is herein incorporated by reference.

### BACKGROUND

People may choose and plan to participate in various exercise regimens to attempt to achieve various goals, such as metabolism increase, athletic-performance enhancement, weight control, maintained or improved cardiovascular function, or overall long-term health.

Choosing and planning an exercise regimen is challenging for two reasons: First, people have different physical capabilities, physical traits, or exercise experiences. Therefore, an exercise regimen that may be particularly suitable for one individual may be much less suitable for another individual.

Second, users may differ in their preference for exercise intensity, or for indoor or outdoor activities. These preferences may even change day-by-day (e.g., depending on how tired a user is or based on the weather).

Thus, there is a need to facilitate generating specifications for workouts that are tuned to individuals' physical capabilities and preferences.

### BRIEF SUMMARY

Some embodiments relate to methods, systems and/or computer-implemented instructions configured to perform or support actions that include: determining a time contribution for each of a set of workout effort zones for a user, wherein each of the set of workout effort zones corresponds to a range of values for a biosignal; determining a timeseries of workout target effort zones for the user based on the target time contributions for the set of workout effort zones; receiving, during a workout time period, real-time biosignal data from a sensor in a wearable electronic device being worn by the user; generating, during the workout time period, an audio, visual, or haptic stimulus based on the real-time biosignal data and a target effort zone in the time series of workout target effort zones; and outputting, during the workout time period, the audio, visual, or haptic stimulus.

Other embodiments of the disclosure are directed to systems, apparatus, and computer readable media associated with methods described herein. In one embodiment, the computer readable medium contains instructions for receiving data and analyzing data, but not instructions for directing a machine to create the data. In another embodiment, the computer readable medium does contain instructions for directing a machine to create the data. In one embodiment, a computer program product comprises a computer readable medium storing a plurality of instructions for controlling a processor to perform an operation for methods described herein. Embodiments are also directed to computer systems configured to perform the steps of any of the methods described herein, potentially with different components performing a respective step or a respective group of steps.

Reference to the remaining portions of the specification, including the drawings and claims, will realize other features and advantages of embodiments of the present disclosure. Further features and advantages, as well as the structure and operation of various embodiments of the present disclosure, are described in detail below with respect to the accompanying drawings. In the drawings, like reference numbers can indicate identical or functionally similar elements.

### BRIEF DESCRIPTION OF THE DRAWINGS

FIG. 1 shows an exemplary process 100 for dynamically defining one or more proposed workout characteristics based on various types on input (e.g., received from a user via a user interface, from sensor data, and/or from data collected from one or more external data sources), generating a proposed workout based on the proposed workout characteristics, and monitoring progress and/or one or more biosignals during the workout in accordance with some embodiments of the disclosure.

FIG. 2 provides one illustration of the input components (e.g., a toggle switch and sliders) that may be provided to receive workout preferences from a user.

FIG. 3 shows the presentation of a subset of biosignals may correspond to a current time minus a predefined preceding offset to a current time plus a predefined subsequent offset.

FIG. 4 shows that an audio feedback stimulus may be provided to recommend effort changes.

FIG. 5 shows an audio feedback stimulus may (e.g., alternatively, or additionally) include a non-verbal stimulus.

FIG. 6 illustrates an alternative collapsed representation of the effort ranges, where the different ranges are stacked horizontally instead of vertically, and prior zones are not shown.

FIG. 7 illustrates a process 1000 for using biosignals to design workouts and provide dynamic feedback to help a user achieve a workout goal.

FIG. 8 is a block diagram of an example electronic device 800.

### DETAILED DESCRIPTION

In some embodiments, one or more characteristics of a proposed workout may be defined based on user input or default values. For example, a workout target effort zone (an example of a characteristic) can be determined based on a time contribution for effort zones. Such a time contribution can be specified by a user or determined by a device worn by the user. A stimulus (e.g., an audio, visual, or haptic stimulus) may be generated that conveys whether the one or more characteristics (e.g., target heart rate value or range) of the proposed workout are being satisfied (e.g., based on measured biosignal data), and the stimulus may be output. As an example, the one or more characteristics may indicate a target biosignal (e.g., a target value of or a target range of a heart rate) of a proposed workout. A workout target effort zone can have a target value of or a target range of the biosignal.

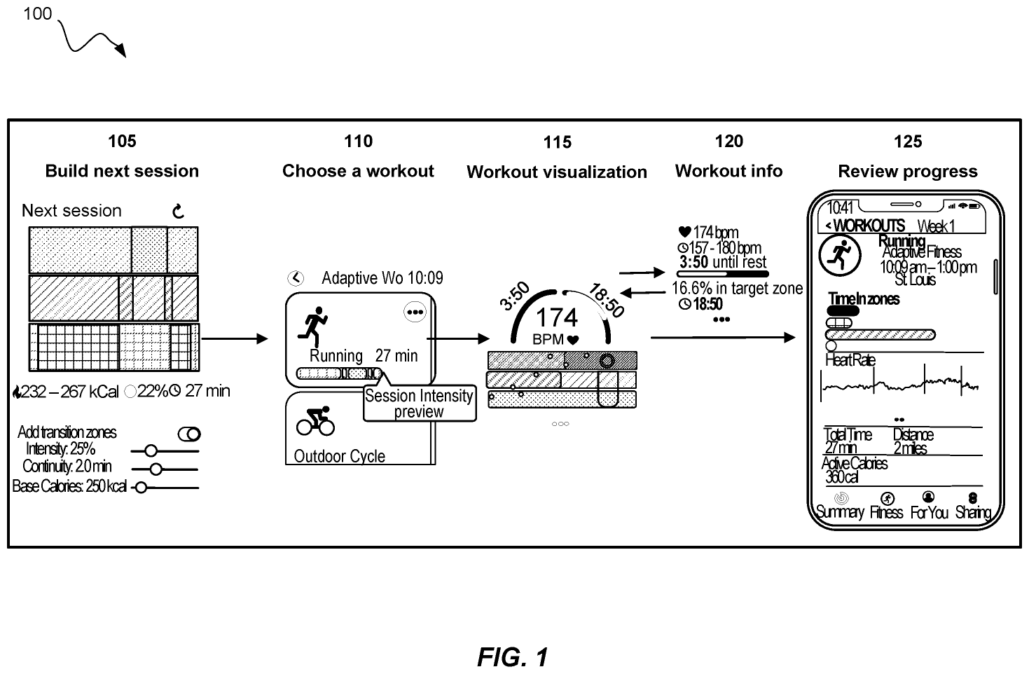

FIG. 1 shows an exemplary process 100 for dynamically defining one or more proposed workout characteristics based on various types on input (e.g., received from a user via a user interface, from sensor data, and/or from data collected from one or more external data sources), generating a proposed workout based on the proposed workout characteristics, and monitoring progress and/or one or more biosignals during the workout in accordance with some embodiments of the disclosure. Part or all of process 100, part or all of each of one or more other processes disclosed herein, part or all of each of one or more actions disclosed herein, and/or part or all of one or more methods disclosed herein may be performed by an electronic device (e.g., worn by a user from whom biosignals are collected and/or outputs are presented). In some instances, an application loaded on the electronic device performs part or all of each of one or more other processes disclosed herein, part or all of each of one or more actions disclosed herein, and/or part or all of one or more methods disclosed herein. The electronic device may be a wearable electronic device (such as a watch, armband, or wristband, etc.).

At block 105, a target time distribution of workout effort zones is determined. Each workout effort zone may be defined based on one or more biosignal metrics. For example, each workout effort zone may correspond to a closed or open range of metrics, wherein the metric is defined based on a current heartrate and a resting heart rate of the user and/or maximum heart rate across heart rates previously observed for the user. The distribution may be determined based on (for example) a preferred workout intensity of the user, and a caloric expenditure target of the user. It will be appreciated that a time contribution, percentage time, or distribution of workout target effort zones (or of target effort zones or of effort zones) can indicate—for each of a set of effort zones—a target absolute amount of time or a target relative amount of time that a biosignal is to be in the zone for a workout. The target time contribution, percentage time, or distribution of workout target effort zones may potentially also identify a target order or a target sequence for which the biosignal is to be within various zones during a workout. For example, a target time contribution may indicate that targets for a biosignal are to be in zone 1 for one minute, followed by zone 2 for 1 minute, followed by zone 1 for 3 minutes. The target time contribution, percentage time, or distribution of workout target effort zones may be defined at a workout-level, and a workout may be defined to include multiple segments. Each segment may be assigned to a target workout effort zone and may have a predefined duration, such that workout-level effort targets (or actual efforts) can be assessed (e.g., by analyzing all of the segments). Some embodiments can assign, to each workout target effort zone of the time series of workout target effort zones, a duration of the workout time period and a particular target range of values for the biosignal from a set of workout effort ranges to the session.

At block 110 of process 100, an electronic device (e.g., a wearable electronic device or another electronic device configured to communicate with a wearable electronic device) receives one or more selections, where each of at least one of the one or more selections corresponds to a selected type of workout. The electronic device may have presented multiple potential selections (e.g., high intensity interval training, hiking, outdoor cycling, outdoor running, stationary cycling, stationary running, etc.).

The multiple potential selections may have been identified by the electronic device or a remote computing system based on (for example) retrieving a pre-identified set of workout-type identifications or identifying or inferring in which workout types the user previously participated. For example, the multiple potential selections may have been identified based on user input that specified workout types of interest or workout types previously performed.

As shown in the exemplary presented potential selection of running, a preview of a time series of target effort zones can be presented so that a user can see how exercise effort during a given session is predicted to progress. In some instances, the time series of target effort zones may be generated based on (for example) a preferred workout intensity of the user, and a caloric expenditure target of the user (see block 105 for an exemplary workout builder interface).

As one illustration of a model: a workout session may include a predefined amount of time in a number of effort zones (e.g., high, medium, and low effort zones). In order to generate this time series of target effort zones, firstly, a base percentage of time in each zone in a workout session is set by using the following thresholds:
Base percentage time in high effort zone (a): User preference
Base percentage time in medium effort zone (b): ½*a*(1−a)
Base percentage time in low effort zone (c): 1−a−b

A session time in minutes may then be defined by relating a base caloric expenditure target of the user to an estimated energy expenditure for a reference time (for example, 60 minutes) of working out within the above effort zone percentages. For example, an estimate for energy expenditure for a workout in the respective effort zones can be generated using a function that depends on one or more user attributes (e.g., biological sex, age, weight, and VO₂ₘₐₓ), as described, for example, in Keytel, L., et al., Prediction of energy expenditure from heart rate monitoring during submaximal exercise. J Sports Sci. 2005; 23(3): 289-297, which is hereby incorporated by reference in its entirety for all purposes.

Once the length of the workout session is determined, a set of effort segments may be generated that vary in length. If segments of a specific effort level would have a duration below a threshold time, these portions of the session may be merged into higher or lower effort zones. The process can be repeated until all effort segments have a length above the threshold time. The segments may then be shuffled. One or more transition segments may then be added to smooth discordant efforts in adjacent effort ranges. Further, adjacent segments having a same effort range may be merged into one, longer segment. As the merger of below-threshold segments and the addition of transition zones will change the overall caloric expenditure of the workout plan, the overall intensity of the plan, and the time spent in each effort zone, the estimated caloric expenditure of the proposed session can be re-adjusted by relating the resulting caloric expenditure to the base caloric expenditure target and re-adjusting the duration of each segment by the resulting factor. The final intensity of the workout plan is determined by the process above and may deviate from the base selection that the user provided. The final energy expenditure estimate for the workout plan can be presented to the user as a range, starting at the estimated energy expenditure for working out at the lower bound of each effort zone, and ending at the estimated energy expenditure for working out at the upper bound of each effort zone.

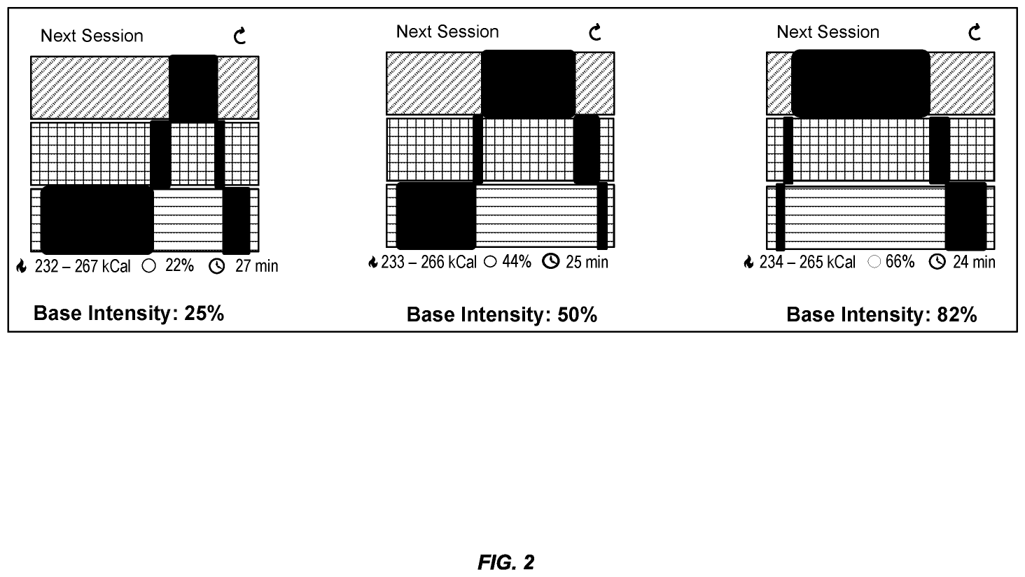

FIG. 2 provides one illustration of the input components (e.g., a toggle switch and sliders) that may be provided to receive workout preferences from a user. As shown, the distribution of target effort ranges changes with the selected base intensity.

In some (additional or alternative) instances, the user may have indicated that a given workout session was preferred by (for example) selecting between multiple possible workout sessions. Each of the possible workout sessions may have been associated with one possible time series of effort zones that resulted from the procedure as described herein.

The device may determine that a workout has begun by detecting a corresponding input from the user (e.g., pressing a “Begin” virtual button or speaking a “Begin workout” command). In some instances, an electronic device performing process 100 continuously collects biosignal data of one or more types so long as the device is being worn by a human. In some instances (e.g., if biosignal data is not continuously collected), an application that performs process 100 can request or instruct collection of such data upon detecting that the workout has begun.

The biosignal data may include (for example) a current heart rate of the user. The biosignal data can be used to predict a real-time effort for the workout for the user, for example, by relating the measured biosignal data to a personal biosignal range of a user (e.g., to a user's resting heart rate and maximum heart rate, as described in Karvonen, J., Vuorimaa, T. Heart Rate and Exercise intensity During Sports Activities. Sports Medicine 5, 303-311 (1988). https://doi.org/10.2165/00007256-198805050-00002, which is hereby incorporated by reference in its entirety for all purposes).

Once it is detected that a workout has begun (block 115), the device may control output (e.g., visual, audio or haptic stimuli) so as to indicate whether an estimated current workout effort (e.g., generated based on one or more detected biosignal measurements) corresponds to a target biosignal and/or a target effort level.

For example, an interface may present a representation of each of one or more target values for and/or one or more detected values for a type of biosignal. As illustrated in block 115, the interface may further present a representation of a range corresponding to each of the one or more target values, each of the one or more detected values, and/or one or more effort ranges.

Representations of the current or historical biosignal values may be superimposed onto representations of one or more effort zones (e.g., as illustrated at block 115). For example, a function may be used to translate a current heart rate (e.g., in relation to a personal range of heart rate values) into an effort level, such that an estimate of a current effort level can be generated based on a recent or current heart rate and the estimated current heart rate can be compared to a target heart rate. As another example, a target heart rate may be estimated by relating a target workout effort to the difference between a user's maximum and resting heartrate and adding their resting heart rate, as described in Karvonen (1988).

As illustrated in block 115, the target effort ranges can be represented via different colored bars having zones with visual dimensions and positions tied to prescribed effort, and the interface can further include information about current or historical biosignal values and overall workout session progress. For example, an elapsed time bar can have an x-offset representing overall session progress. Target zones can have visual dimensions and positions, a point representing a current effort, estimated, e.g., from heart rate. can have a position, and points representing historical effort can have positions as follows:

On visual update:
// overall dimensions
timelineWidth = screenWidth − 2 * baseIndicatorSize // align leading
timelineHeight = screenHeight − 2* baseIndicatorSize // align center
// visualize session progress
elapsedTimeBarWidth = baseIndicatorSize
elapsedTimeBarHeight = timelineHeight + 2 * baseIndicatorSize
elapsedTimeBarX (offset) = timelineWidth * currentProgress
elapsedTimeBarY (offset) = − baseIndicatorSize
// visualize target zones
for zone in target zones:
zoneWidth = (zoneStart − zoneEnd) * baseZoneWidth
zoneHeight = timelineHeight
* ( (zoneUpperTarget − sessionMinTarget) /
(sessionMaxTarget − sessionMinTarget)
− (zoneLowerTarget − sessionMinTarget) /
(sessionMaxTarget − sessionMinTarget))
zoneX (offset) = timelineWidth * currentProgress
+ baseZoneWidth * zoneStart
+ timelineWidth * currentProgress
− baseZoneWidth * currentProgress
zoneY (offset) = timelineHeight − zoneHeight
− timelineHeight * (zoneLowerTarget − sessionMinTarget) /
(sessionMaxTarget −
sessionMinTarget)
// visualize collected effort estimates, e.g., from heart rate
for (effort, progress) in collected (effort, progress) estimates:
// visualize the most recent effort estimate
if current:
effortSize(width, height) = baseIndicatorSize
effortX(offset) = timelineWidth * currentProgress
effortY(offset) = timelineHeight
* (1 − ((effort − sessionMinTarget) /
(sessionMaxTarget − sessionMinTarget)))
− baseIndicatorSize // clipped to 0...1
// visualize previous estimates
else:
effortSize(width, height) = baseIndicatorSize / 4
effortX(offset) = timelineWidth * progress +
baseZoneWidth * (progress − currentProgress)
effortY(offset) = timelineHeight
* (1 − ((effort − sessionMinTarget) /
(sessionMaxTarget − sessionMinTarget)))
− baseIndicatorSize / 4 // clipped to 0...1

where screen width, height, timeline width, height, zone width, height, X offsets, and Y offsets are measured in screen units (e.g., points, pixels); progress, zone start, and zone end range from 0 to 100% of the total session time; zone targets and estimated effort range from 0 to 100% effort (e.g., estimates from heart rate, as described above); X and Y offsets are computed with timeline width and height as reference frame; effort Y offsets are clamped between 0 and 100% of the timeline height; baseZoneWidth and baseIndicatorSize are visual constants, set in screen units (e.g., points, pixels).

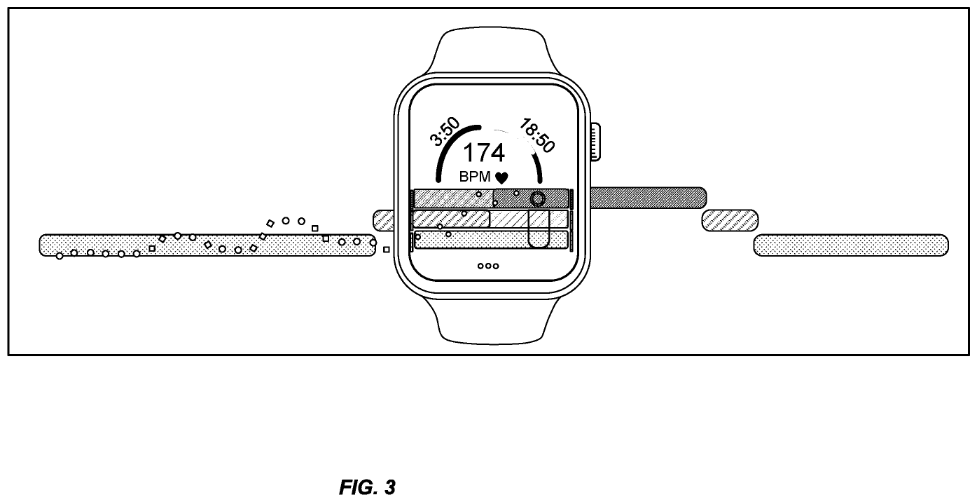

It will be appreciated that a given presentation of a historical, current and/or target biosignal (and/or target effort zone(s)) may be a subset of those that are captured across a session. As illustrated in FIG. 3, the subset may correspond to a current time minus a predefined preceding offset to a current time plus a predefined subsequent offset. In various circumstances, these visual offsets may be dynamically adjusted (e.g. by changing baseZoneWidth). Accordingly, each workout target effort zone in the time series of workout target effort zones can include a workout target effort zone from among the set of workout target effort zones, where the audio, visual, or haptic stimulus identifies a particular target workout target effort zone corresponding to a current time.

The interface may indicate whether a current effort level that is estimated based on one or more observed biosignal values matches (or is sufficiently close to) a current target effort level and/or may identify a current effort level. For example, at block 115, a representation of current biosignal is presented by the large circle (174 bpm heart rate, transformed to effort), along with a representation of a target effort zone (high, as the translucent bar representing overall workout session progress is hovering over the orange, high effort zone). Historical biosignal values, transformed to effort, are represented by smaller, white circles. In the illustrated scenario, vertical locations of the small circle, large circle, and bars are determined by effort, as computed by the equations described above. The horizontal locations of these elements are determined by session progress.

At block 115, the target effort zone and the inferred current effort level (as inferred based on one or more detected biosignals) are the same. It will be appreciated that in the same, similar, or contrary circumstances, the device may output stimuli that indicate whether and/or an extent to which the inferred effort level matches (or differs from) a target effort level. For example, a visual presentation may vertically position one representation of a target effort level (for example, as a horizontal bar, as described above) and vertically position another representation of inferred current effort level (for example, as large circle, as described above).

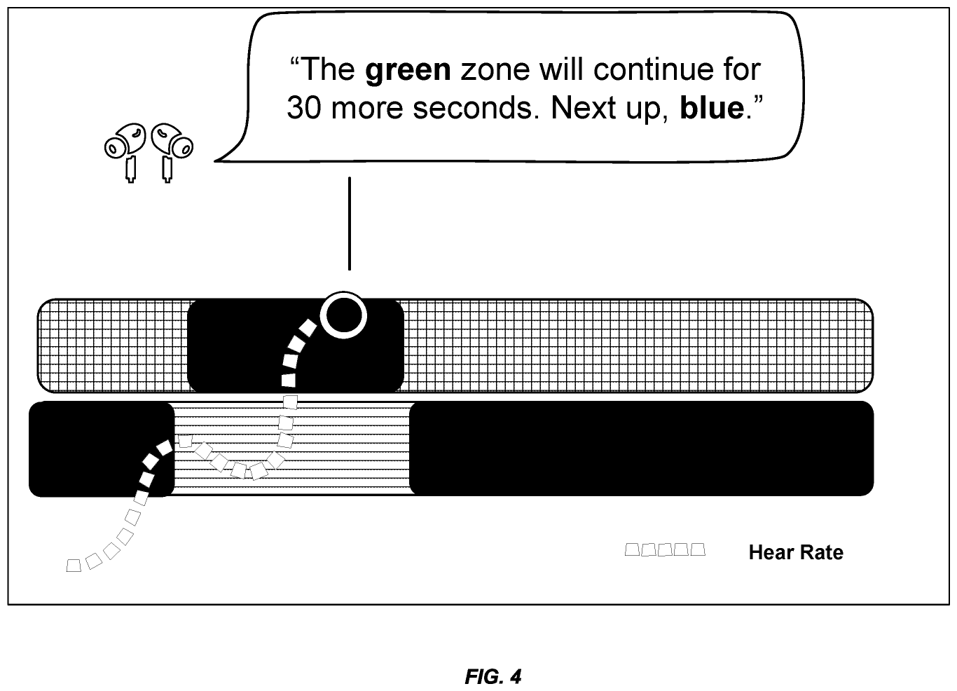

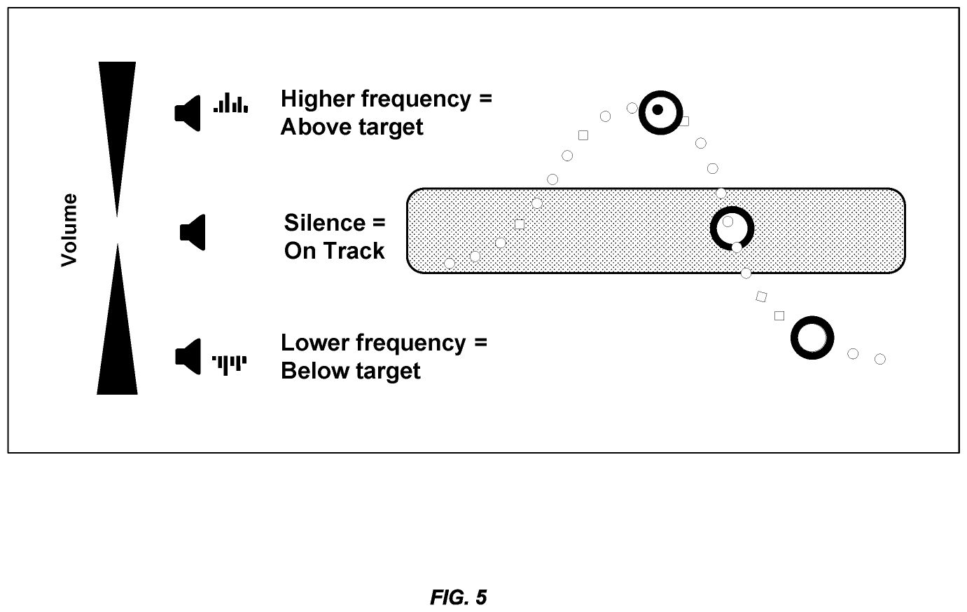

As another example, an audio stimulus may be generated such that one or several acoustic properties of the stimulus vary (e.g., monotonically vary) based on a difference between the target effort level and the inferred current effort level. To illustrate, a volume of a looping audio feedback stimulus may monotonically depend on an extent to which a target effort level differs from an inferred current effort level. As another illustration, an audio feedback stimulus may be provided to recommend effort changes (e.g., as illustrated in FIG. 4 and FIG. 5). As illustrated in FIG. 4, an audio feedback stimulus may include speech (e.g., simulated speech) that may identify a current effort range of the user, a current target effort range generated for the user, and/or an upcoming target effort range.

As illustrated in FIG. 5, an audio feedback stimulus may (e.g., alternatively, or additionally) include a non-verbal stimulus (e.g., a looping audio sample, beep, tone, audible pulse, etc.) where acoustic properties of the stimulus are indicative of an extent to which an inferred current effort of the user differs from a current target effort range. For example, the volume of a non-verbal audio stimulus may scale with an extent to which a user's current effort is above or below a current target effort range, as defined below. Differences are measured from the upper limit of a target effort zone for audio feedback on inferred current effort above the target effort zone, and from the lower limit of a target effort zone for audio feedback on inferred current effort below the target effort zone. In some instances, there is no non-verbal audio stimulus when a user's current effort is within the current target effort range. Example pseudocode is as follows:

On new effort estimate:
if effort > zoneUpperTarget:
currentFeedbackVolume = abs(effort − zoneUpperTarget) /
maxDifference * maxVolume
else if effort < zoneLowerTarget:
currentFeedbackVolume = abs(effort − zoneLowerTarget) /
maxDifference * maxVolume
else:
currentFeedbackVolume = 0
where abs(effort-target) is clamped between 0 and maxDifference;
and maxDifference and maxVolume are constants.

In some embodiments, the extent to which a current effort level of the user differs from the workout target effort zone can be estimated, based on the real-time biosignal data and at least part of the time series of workout target effort zone. A stimulus property (e.g., the position of a volume) based determined based on the estimated extent. The audio, visual, or haptic stimulus can be generated based on the stimulus property. As part of determining the extent, a current value of the biosignal can be compared to the target range of values for the biosignal of the workout target effort zone.

Returning to FIG. 1, block 120 illustrates how a user can toggle a screen to track their progress with workout related or absolute data. For example, workout-related data may identify an estimated or actual duration of time in a workout; an indication of an upcoming zone; an explicit identification of a current, past, or target biosignal value, a distance to a target, etc.

Lastly, block 125 illustrates how a user can review information corresponding to a completed workout session. The interface may include, for example, a time series of measured biosignal values, average pace and distance, as well as absolute time or percentage time spent in each effort zone. The interface may also allow a user to review a previous workout session.

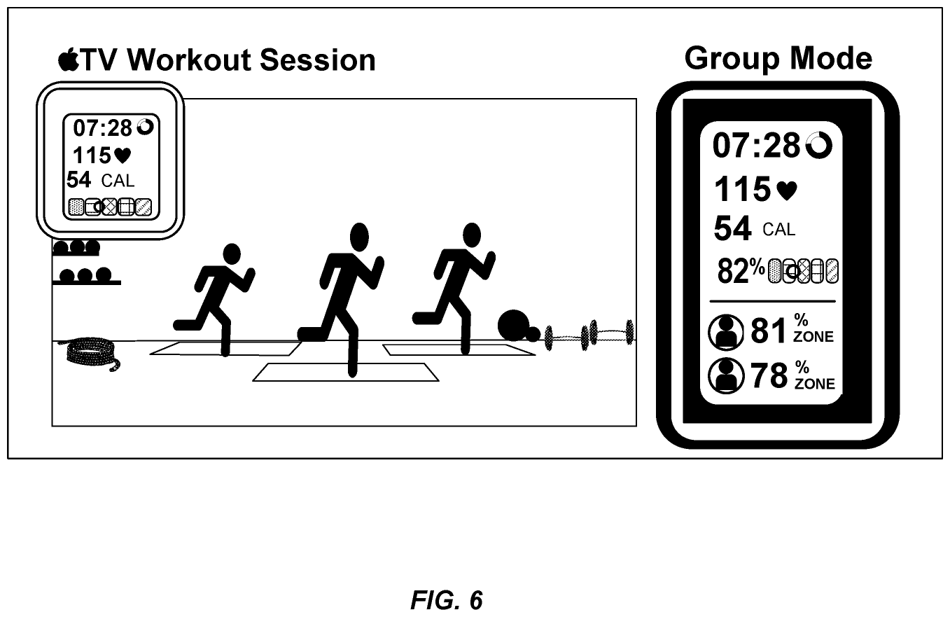

It will be appreciated that additional or alternative functionalities are contemplated relative to those disclosed. For example, FIG. 6 illustrates an alternative collapsed representation of the effort ranges, where the different ranges are stacked horizontally instead of vertically, and prior zones are not shown. This collapsed representation may facilitate showing a larger amount of other types of data on a screen. As another example, FIG. 6 illustrates that the technique may be used while performing a workout at a single location (e.g., and not outside). Such workouts may include following workout instructions provided on a television or computer screen, using a treadmill, using a stationary bike, etc. As yet another example, an interface may be configured to present “group mode” data where a given user may be able to see real-time information as to in which effort zone (or at what effort level) one or more contacts are, or at in what percentage of the workout session the effort level of one or more contacts has matched the target effort zone. Such information may also be transmitted from the user's device to the one or more contacts.

Thus, embodiments disclosed herein facilitate using dynamic data and various models to provide workout structures and feedbacks that can help a user achieve a workout goal. The effort-level zones and dynamic feedback can help a user to easily internalize how to modify a workout effort (in real-time) to meet the workout goal.

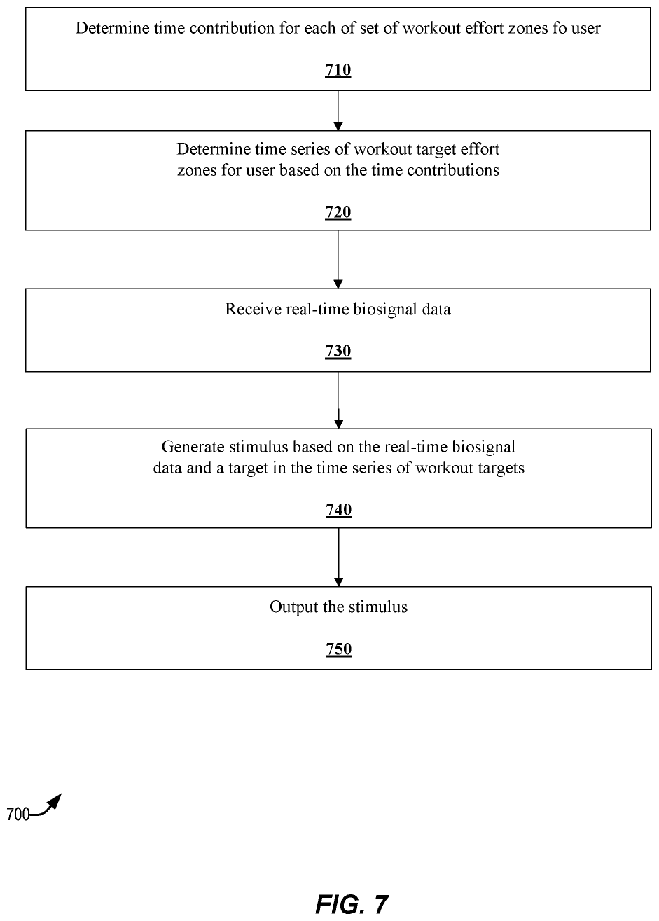

FIG. 7 illustrates a process 1000 for using biosignals to design workouts and provide dynamic feedback to help a user achieve a workout goal. Part or all of process 700 may be performed at a user device, such as a wearable device. It will be appreciated that the blocks in process 700 correspond to select actions relating to embodiments of the disclosure. It will be appreciated that disclosures presented above may pertain further details pertaining to one or more of the blocks 700; may illustrate how one or more blocks of process 700 may be modified; and/or may illustrate how process 700 may include fewer or more actions than what is depicted in FIG. 7.

At block 710, a time contribution for each of a set of workout effort zones is determined for a user. The determination may be based on (for example) a preferred workout intensity of the user, and a caloric expenditure target of the user. A time contribution for a workout effort zone may identify a target absolute or relative amount of time that a biosignal of a user is to correspond to a given effort zone. A given time contribution may be specific to a given time interval or cumulative. For example, block 710 may include determining a target sequence of workout effort zones, where the sequence includes an ordered identification of effort zones and a duration for each zone. As another example, block 710 may include identifying an absolute amount of cumulative time in a workout or a target percentage of time in a workout that is to be allocated to a given target workout effort zone. Each of the workout effort zone percentage times corresponds to a particular target range of values for a biosignal. The biosignal(s) may include any biosignal disclosed herein, such as a heart rate. A total session time is generated by relating the expected caloric burn in each effort zone to a user's caloric burn base goal.

At block 720, a time series of target effort zones for the user is determined based on the set of workout effort zone percentage times and the total session time. In some instances, a series of workout sessions are defined, where each session is to correspond to one possible time series of effort zones.

At block 730, real-time biosignal data is received during a workout period. The real-time biosignal data can be received from and/or may have been collected by a sensor in the user device. The user device can include wearable electronic device being worn by the user.

At block 740, during the workout time period, an audio, visual, or haptic stimulus is generated based on the real-time biosignal data and a target effort zone in the time series of workout target effort zones. The stimulus may (for example) indicate how closely a detected biosignal corresponds to a target biosignal (or target effort range), may recommend a particular change (e.g., to increase a speed) to the user, etc.

At block 750 the audio, visual, or haptic stimulus is output.

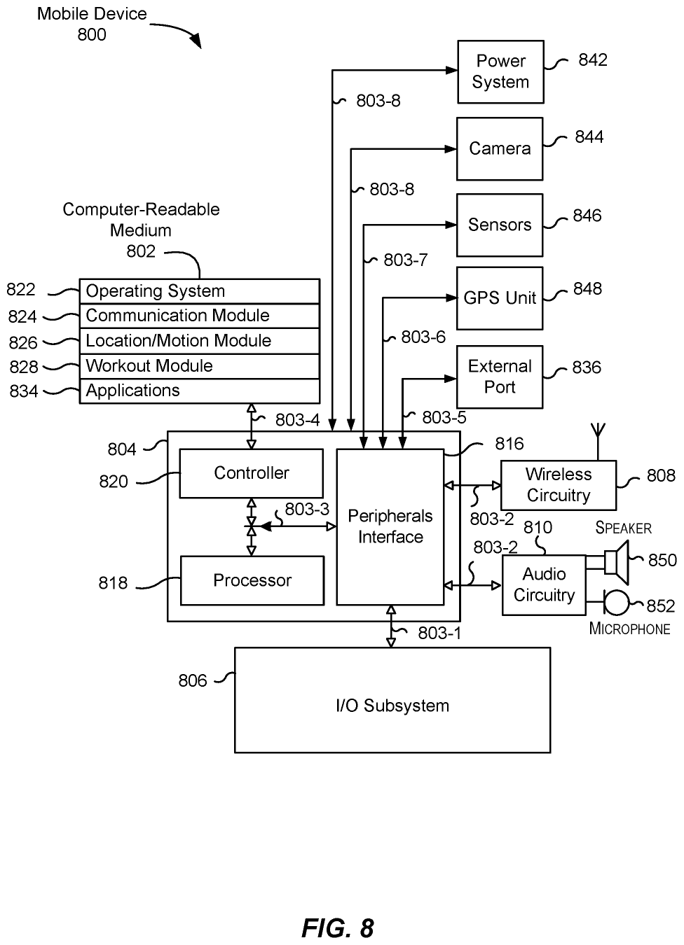

FIG. 8 is a block diagram of an example electronic device 800 also referred to as a computing device. Device 800 generally includes computer-readable medium 802, a processing system 804, an Input/Output (I/O) subsystem 806, wireless circuitry 808, and audio circuitry 810 including speaker 812 and microphone 814. These components may be coupled by one or more communication buses or signal lines 803. Device 800 can be any portable electronic device, including a handheld computer, a tablet computer, a mobile phone, laptop computer, tablet device, media player, personal digital assistant (PDA), a key fob, a car key, an access card, a multifunction device, a mobile phone, a portable gaming device, a headset, or the like, including a combination of two or more of these items.

It should be apparent that the architecture shown in FIG. 8 is only one example of an architecture for device 800, and that device 800 can have more or fewer components than shown, or a different configuration of components. The various components shown in FIG. 8 can be implemented in hardware, software, or a combination of both hardware and software, including one or more signal processing and/or application specific integrated circuits.

Wireless circuitry 808 is used to send and receive information over a wireless link or network to one or more other devices' conventional circuitry such as an antenna system, a radio frequency (RF) transceiver, one or more amplifiers, a tuner, one or more oscillators, a digital signal processor, a coder-decoder (CODEC) chipset, memory, etc. Wireless circuitry 808 can use various protocols, e.g., as described herein. In various embodiments, wireless circuitry 808 is capable of establishing and maintaining communications with other devices using one or more communication protocols, including time division multiple access (TDMA), code division multiple access (CDMA), global system for mobile communications (GSM), Enhanced Data GSM Environment (EDGE), wideband code division multiple access (W-CDMA), Long Term Evolution (LTE), LTE-Advanced, Wi-Fi (such as Institute of Electrical and Electronics Engineers (IEEE) 802.11a, IEEE 802.11b, IEEE 802.11g and/or IEEE 802.11n), Bluetooth, Wi-MAX, Voice Over Internet Protocol (VoIP), near field communication protocol (NFC), a protocol for email, instant messaging, and/or a short message service (SMS), or any other suitable communication protocol, including communication protocols not yet developed as of the filing date of this document.

Wireless circuitry 808 is coupled to processing system 804 via peripherals interface 816. Peripherals interface 816 can include conventional components for establishing and maintaining communication between peripherals and processing system 804. Voice and data information received by wireless circuitry 808 (e.g., in speech recognition or voice command applications) is sent to one or more processors 818 via peripherals interface 816. One or more processors 818 are configurable to process various data formats for one or more application programs 834 stored on medium 802.

Peripherals interface 816 couple the input and output peripherals of device 800 to the one or more processors 818 and computer-readable medium 802. One or more processors 818 communicate with computer-readable medium 802 via a controller 820. Computer-readable medium 802 can be any device or medium that can store code and/or data for use by one or more processors 818. Computer-readable medium 802 can include a memory hierarchy, including cache, main memory and secondary memory. The memory hierarchy can be implemented using any combination of a random-access memory (RAM) (e.g., static random access memory (SRAM,) dynamic random access memory (DRAM), double data random access memory (DDRAM)), read only memory (ROM), FLASH, magnetic and/or optical storage devices, such as disk drives, magnetic tape, CDs (compact disks) and DVDs (digital video discs). In some embodiments, peripherals interface 816, one or more processors 818, and controller 820 can be implemented on a single chip, such as processing system 804. In some other embodiments, they can be implemented on separate chips.

Processor(s) 818 can include hardware and/or software elements that perform one or more processing functions, such as mathematical operations, logical operations, data manipulation operations, data transfer operations, controlling the reception of user input, controlling output of information to users, or the like. Processor(s) 818 can be embodied as one or more hardware processors, microprocessors, microcontrollers, field programmable gate arrays (FPGAs), application-specified integrated circuits (ASICs), or the like.

Device 800 also includes a power system 842 for powering the various hardware components. Power system 842 can include a power management system, one or more power sources (e.g., battery, alternating current (AC)), a recharging system, a power failure detection circuit, a power converter or inverter, a power status indicator (e.g., a light emitting diode (LED)) and any other components typically associated with the generation, management and distribution of power in mobile devices.

In some embodiments, device 800 includes a camera 844. In some embodiments, device 800 includes sensors 846. Sensors can include accelerometers, compass, gyrometer, pressure sensors, audio sensors, light sensors, barometers, and the like. Sensors 846 can be used to sense location aspects, such as auditory or light signatures of a location.

In some embodiments, device 800 can include a GPS receiver, sometimes referred to as a GPS unit 848. A mobile device can use a satellite navigation system, such as the Global Positioning System (GPS), to obtain position information, timing information, altitude, or other navigation information. During operation, the GPS unit can receive signals from GPS satellites orbiting the Earth. The GPS unit analyzes the signals to make a transit time and distance estimation. The GPS unit can determine the current position (current location) of the mobile device. Based on these estimations, the mobile device can determine a location fix, altitude, and/or current speed. A location fix can be geographical coordinates such as latitudinal and longitudinal information.

One or more processors 818 run various software components stored in medium 802 to perform various functions for device 800. In some embodiments, the software components include an operating system 822, a communication module 824 (or set of instructions), a location module 826 (or set of instructions), a workout module 828 that is used as part of an adaptive workout operation described herein, and other application programs 834 (or set of instructions).

Operating system 822 can be any suitable operating system, including iOS, Mac OS, Darwin, Real Time Operating System (RTXC), LINUX, UNIX, OS X, WINDOWS, or an embedded operating system such as VxWorks. The operating system can include various procedures, sets of instructions, software components and/or drivers for controlling and managing general system tasks (e.g., memory management, storage device control, power management, etc.) and facilitates communication between various hardware and software components.

Communication module 824 facilitates communication with other devices over one or more external ports 836 or via wireless circuitry 808 and includes various software components for handling data received from wireless circuitry 808 and/or external port 836. External port 836 (e.g., universal serial bus (USB), FireWire, Lightning connector, 60-pin connector, etc.) is adapted for coupling directly to other devices or indirectly over a network (e.g., the Internet, wireless local area network (LAN), etc.).

Location/motion module 826 can assist in determining the current position (e.g., coordinates or other geographic location identifiers) and motion of device 800. Modern positioning systems include satellite based positioning systems, such as Global Positioning System (GPS), cellular network positioning based on “cell IDs,” and Wi-Fi positioning technology based on a Wi-Fi networks. GPS also relies on the visibility of multiple satellites to determine a position estimate, which may not be visible (or have weak signals) indoors or in “urban canyons.” In some embodiments, location/motion module 826 receives data from GPS unit 848 and analyzes the signals to determine the current position of the mobile device. In some embodiments, location/motion module 826 can determine a current location using Wi-Fi or cellular location technology. For example, the location of the mobile device can be estimated using knowledge of nearby cell sites and/or Wi-Fi access points with knowledge also of their locations. Information identifying the Wi-Fi or cellular transmitter is received at wireless circuitry 808 and is passed to location/motion module 826. In some embodiments, the location module receives the one or more transmitter IDs. In some embodiments, a sequence of transmitter IDs can be compared with a reference database (e.g., Cell ID database, Wi-Fi reference database) that maps or correlates the transmitter IDs to position coordinates of corresponding transmitters, and computes estimated position coordinates for device 800 based on the position coordinates of the corresponding transmitters. Regardless of the specific location technology used, location/motion module 826 receives information from which a location fix can be derived, interprets that information, and returns location information, such as geographic coordinates, latitude/longitude, or other location fix data.

Workout module 828 can send/receive ranging messages to/from an antenna, e.g., connected to wireless circuitry 808. The messages can be used for various purposes, e.g., to identify a sending antenna of a device, determine timestamps of messages to determine a distance of mobile device 800 from another device. Ranging module 828 can exist on various processors of the device, e.g., an always-on processor (AOP), a UWB chip, and/or an application processor. For example, parts of ranging module 828 can determine a distance on an AOP, and another part of the ranging module can interact with a sharing module, e.g., to display a position of the other device on a screen in order for a user to select the other device to share a data item. Ranging module 828 can also interact with a reminder module that can provide an alert based on a distance from another mobile device.

The one or more applications 834 on device 800 can include any applications installed on the device 800, including without limitation, a browser, address book, contact list, email, instant messaging, social networking, word processing, keyboard emulation, widgets, JAVA-enabled applications, encryption, digital rights management, voice recognition, voice replication, a music player (which plays back recorded music stored in one or more files, such as MP3 or AAC files), etc.

There may be other modules or sets of instructions (not shown), such as a graphics module, a time module, etc. For example, the graphics module can include various conventional software components for rendering, animating and displaying graphical objects (including without limitation text, web pages, icons, digital images, animations and the like) on a display surface. In another example, a timer module can be a software timer. The timer module can also be implemented in hardware. The time module can maintain various timers for any number of events.

I/O subsystem 806 can be coupled to a display system (not shown), which can be a touch-sensitive display. The display displays visual output to the user in a graphical user interface (GUI). The visual output can include text, graphics, video, and any combination thereof. Some or all of the visual output can correspond to user-interface objects. A display can use LED (light emitting diode), LCD (liquid crystal display) technology, or LPD (light emitting polymer display) technology, although other display technologies can be used in other embodiments.

In some embodiments, I/O subsystem 806 can include a display and user input devices such as a keyboard, mouse, and/or trackpad. In some embodiments, I/O subsystem 806 can include a touch-sensitive display. A touch-sensitive display can also accept input from the user based at least part on haptic and/or tactile contact. In some embodiments, a touch-sensitive display forms a touch-sensitive surface that accepts user input. The touch-sensitive display/surface (along with any associated modules and/or sets of instructions in computer-readable medium 802) detects contact (and any movement or release of the contact) on the touch-sensitive display and converts the detected contact into interaction with user-interface objects, such as one or more soft keys, that are displayed on the touch screen when the contact occurs. In some embodiments, a point of contact between the touch-sensitive display and the user corresponds to one or more digits of the user. The user can make contact with the touch-sensitive display using any suitable object or appendage, such as a stylus, pen, finger, and so forth. A touch-sensitive display surface can detect contact and any movement or release thereof using any suitable touch sensitivity technologies, including capacitive, resistive, infrared, and surface acoustic wave technologies, as well as other proximity sensor arrays or other elements for determining one or more points of contact with the touch-sensitive display.

Further, I/O subsystem 806 can be coupled to one or more other physical control devices (not shown), such as pushbuttons, keys, switches, rocker buttons, dials, slider switches, sticks, LEDs, etc., for controlling or performing various functions, such as power control, speaker volume control, ring tone loudness, keyboard input, scrolling, hold, menu, screen lock, clearing and ending communications and the like. In some embodiments, in addition to the touch screen, device 800 can include a touchpad (not shown) for activating or deactivating particular functions. In some embodiments, the touchpad is a touch-sensitive area of the device that, unlike the touch screen, does not display visual output. The touchpad can be a touch-sensitive surface that is separate from the touch-sensitive display, or an extension of the touch-sensitive surface formed by the touch-sensitive display.

In some embodiments, some or all of the operations described herein can be performed using an application executing on the user's device. Circuits, logic modules, processors, and/or other components may be configured to perform various operations described herein. Those skilled in the art will appreciate that, depending on implementation, such configuration can be accomplished through design, setup, interconnection, and/or programming of the particular components and that, again depending on implementation, a configured component might or might not be reconfigurable for a different operation. For example, a programmable processor can be configured by providing suitable executable code; a dedicated logic circuit can be configured by suitably connecting logic gates and other circuit elements; and so on.

Any of the software components or functions described in this application, may be implemented as software code to be executed by a processor using any suitable computer language such as, for example, Java, C++ or Perl using, for example, conventional or object-oriented techniques. The software code may be stored as a series of instructions, or commands on a computer readable medium for storage and/or transmission, suitable media include random access memory (RAM), a read only memory (ROM), a magnetic medium such as a hard-drive or a floppy disk, or an optical medium such as a compact disk (CD) or DVD (digital versatile disk), flash memory, and the like. The computer readable medium may be any combination of such storage or transmission devices.

Such programs may also be encoded and transmitted using carrier signals adapted for transmission via wired, optical, and/or wireless networks conforming to a variety of protocols, including the Internet. As such, a computer readable medium according to an embodiment of the present disclosure may be created using a data signal encoded with such programs. Computer readable media encoded with the program code may be packaged with a compatible device or provided separately from other devices (e.g., via Internet download). Any such computer readable medium may reside on or within a single computer program product (e.g., a hard drive or an entire computer system), and may be present on or within different computer program products within a system or network. A computer system may include a monitor, printer, or other suitable display for providing any of the results mentioned herein to a user.

Computer programs incorporating various features of the present disclosure may be encoded on various computer readable storage media; suitable media include magnetic disk or tape, optical storage media, such as compact disk (CD) or DVD (digital versatile disk), flash memory, and the like. Computer readable storage media encoded with the program code may be packaged with a compatible device or provided separately from other devices. In addition, program code may be encoded and transmitted via wired optical, and/or wireless networks conforming to a variety of protocols, including the Internet, thereby allowing distribution, e.g., via Internet download. Any such computer readable medium may reside on or within a single computer product (e.g., a solid-state drive, a hard drive, a CD, or an entire computer system), and may be present on or within different computer products within a system or network. A computer system may include a monitor, printer, or other suitable display for providing any of the results mentioned herein to a user.

As described above, one aspect of the present technology is the gathering, sharing, and use of data, including an authentication tag and data from which the tag is derived. The present disclosure contemplates that in some instances, this gathered data may include personal information data that uniquely identifies or can be used to contact or locate a specific person. Such personal information data can include demographic data, location-based data, telephone numbers, email addresses, twitter ID's, home addresses, data or records relating to a user's health or level of fitness (e.g., vital signs measurements, medication information, exercise information), date of birth, or any other identifying or personal information.

The present disclosure recognizes that the use of such personal information data, in the present technology, can be used to the benefit of users. For example, the personal information data can be used to authenticate another device, and vice versa to control which devices ranging operations may be performed. Further, other uses for personal information data that benefit the user are also contemplated by the present disclosure. For instance, health and fitness data may be shared to provide insights into a user's general wellness, or may be used as positive feedback to individuals using technology to pursue wellness goals.

The present disclosure contemplates that the entities responsible for the collection, analysis, disclosure, transfer, storage, or other use of such personal information data will comply with well-established privacy policies and/or privacy practices. In particular, such entities should implement and consistently use privacy policies and practices that are generally recognized as meeting or exceeding industry or governmental requirements for maintaining personal information data private and secure. Such policies should be easily accessible by users and should be updated as the collection and/or use of data changes. Personal information from users should be collected for legitimate and reasonable uses of the entity and not shared or sold outside of those legitimate uses. Further, such collection/sharing should occur after receiving the informed consent of the users. Additionally, such entities should consider taking any needed steps for safeguarding and securing access to such personal information data and ensuring that others with access to the personal information data adhere to their privacy policies and procedures. Further, such entities can subject themselves to evaluation by third parties to certify their adherence to widely accepted privacy policies and practices. In addition, policies and practices should be adapted for the particular types of personal information data being collected and/or accessed and adapted to applicable laws and standards, including jurisdiction-specific considerations. For instance, in the US, collection of or access to certain health data may be governed by federal and/or state laws, such as the Health Insurance Portability and Accountability Act (HIPAA); whereas health data in other countries may be subject to other regulations and policies and should be handled accordingly. Hence different privacy practices should be maintained for different personal data types in each country.

Despite the foregoing, the present disclosure also contemplates embodiments in which users selectively block the use of, or access to, personal information data. That is, the present disclosure contemplates that hardware and/or software elements can be provided to prevent or block access to such personal information data. For example, in the case of sharing content and performing ranging, the present technology can be configured to allow users to select to “opt in” or “opt out” of participation in the collection of personal information data during registration for services or anytime thereafter. In addition to providing “opt in” and “opt out” options, the present disclosure contemplates providing notifications relating to the access or use of personal information. For instance, a user may be notified upon downloading an app that their personal information data will be accessed and then reminded again just before personal information data is accessed by the app.

Moreover, it is the intent of the present disclosure that personal information data should be managed and handled in a way to minimize risks of unintentional or unauthorized access or use. Risk can be minimized by limiting the collection of data and deleting data once it is no longer needed. In addition, and when applicable, including in certain health related applications, data de-identification can be used to protect a user's privacy. De-identification may be facilitated, when appropriate, by removing specific identifiers (e.g., date of birth, etc.), controlling the amount or specificity of data stored (e.g., collecting location data a city level rather than at an address level), controlling how data is stored (e.g., aggregating data across users), and/or other methods.

Therefore, although the present disclosure broadly covers use of personal information data to implement one or more various disclosed embodiments, the present disclosure also contemplates that the various embodiments can also be implemented without the need for accessing such personal information data. That is, the various embodiments of the present technology are not rendered inoperable due to the lack of all or a portion of such personal information data.

Although the present disclosure has been described with respect to specific embodiments, it will be appreciated that the disclosure is intended to cover all modifications and equivalents within the scope of the following claims.

All patents, patent applications, publications, and descriptions mentioned herein are incorporated by reference in their entirety for all purposes. None is admitted to be prior art.

The specification and drawings are, accordingly, to be regarded in an illustrative rather than a restrictive sense. It will, however, be evident that various modifications and changes may be made thereunto without departing from the broader spirit and scope of the disclosure as set forth in the claims.

Other variations are within the spirit of the present disclosure. Thus, while the disclosed techniques are susceptible to various modifications and alternative constructions, certain illustrated embodiments thereof are shown in the drawings and have been described above in detail. It should be understood, however, that there is no intention to limit the disclosure to the specific form or forms disclosed, but on the contrary, the intention is to cover all modifications, alternative constructions and equivalents falling within the spirit and scope of the disclosure, as defined in the appended claims.

The use of the terms “a” and “an” and “the” and similar referents in the context of describing the disclosed embodiments (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. The term “connected” is to be construed as partly or wholly contained within, attached to, or joined together, even if there is something intervening. The phrase “based on” should be understood to be open-ended, and not limiting in any way, and is intended to be interpreted or otherwise read as “based at least in part on,” where appropriate. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate embodiments of the disclosure and does not pose a limitation on the scope of the disclosure unless otherwise claimed. No language in the specification should be construed as indicating any non-claimed element as essential to the practice of the disclosure. The use of “or” is intended to mean an “inclusive or,” and not an “exclusive or” unless specifically indicated to the contrary. Reference to a “first” component does not necessarily require that a second component be provided. Moreover, reference to a “first” or a “second” component does not limit the referenced component to a particular location unless expressly stated. The term “based on” is intended to mean “based at least in part on.”

Disjunctive language such as the phrase “at least one of X, Y, or Z,” unless specifically stated otherwise, is otherwise understood within the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present. Additionally, conjunctive language such as the phrase “at least one of X, Y, and Z,” unless specifically stated otherwise, should also be understood to mean X, Y, Z, or any combination thereof, including “X, Y, and/or Z.”

Preferred embodiments of this disclosure are described herein, including the best mode known to the inventors for carrying out the disclosure. Variations of those preferred embodiments may become apparent to those of ordinary skill in the art upon reading the foregoing description. The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

The specific details of particular embodiments may be combined in any suitable manner or varied from those shown and described herein without departing from the spirit and scope of embodiments of the disclosure.

The above description of exemplary embodiments of the disclosure has been presented for the purposes of illustration and description. It is not intended to be exhaustive or to limit the disclosure to the precise form described, and many modifications and variations are possible in light of the teaching above. The embodiments were chosen and described in order to best explain the principles of the disclosure and its practical applications to thereby enable others skilled in the art to best utilize the disclosure in various embodiments and with various modifications as are suited to the particular use contemplated.

All references, including publications, patent applications, and patents, cited herein are hereby incorporated by reference to the same extent as if each reference were individually and specifically indicated to be incorporated by reference and were set forth in its entirety herein.

What is claimed is:

1. A computer-implemented method comprising:
determining a target time contribution for each of a set of workout target effort zones for a workout of a user, thereby determining target time contributions, wherein each of the set of workout target effort zones corresponds to a target range of values for a biosignal;
determining a time series of workout target effort zones for the user based on the target time contributions for the set of workout target effort zones;
receiving, during a workout time period, real-time biosignal data from a sensor in a wearable electronic device being worn by the user;
generating, during the workout time period, an audio, visual, or haptic stimulus based on the real-time biosignal data and a workout target effort zone in the time series of workout target effort zones; and
outputting, during the workout time period, the audio, visual, or haptic stimulus.

2. The computer-implemented method of claim 1, wherein the biosignal is a heart rate.

3. The computer-implemented method of claim 1, wherein each workout target effort zone in the time series of workout target effort zones includes a workout target effort zone from among the set of workout target effort zones, and wherein the audio, visual, or haptic stimulus identifies a particular workout target effort zone corresponding to a current time.

4. The computer-implemented method of claim 1, further comprising:
estimating, based on the real-time biosignal data and at least part of the time series of workout target effort zones, an extent to which a current effort level of the user differs from the workout target effort zone; and
determining a stimulus property based on the extent, wherein the audio, visual, or haptic stimulus is generated based on the stimulus property.

5. The computer-implemented method of claim 4, wherein estimating, based on the real-time biosignal data and at least part of the time series of workout target effort zones, the extent to which the current effort level of the user differs from the workout target effort zone includes:
comparing a current value of the biosignal to the target range of values for the biosignal of the workout target effort zone.

6. The computer-implemented method of claim 1, wherein the audio, visual, or haptic stimulus includes an audio stimulus.

7. The computer-implemented method of claim 1, wherein determining the time series of workout target effort zones for the user based on the target time contributions for the set of workout target effort zones comprises using a model that transforms the biosignal to an estimated energy expenditure.

8. The computer-implemented method of claim 1, wherein determining the time series of workout target effort zones includes:
determining a target percentage of time to be spent across the set of workout target effort zones based on one or more preferences identified by the user;
defining a set of sessions for the workout, where each session corresponds to the time series of workout target effort zones; and
assigning, to each workout target effort zone of the time series of workout target effort zones, a duration of the workout time period and a particular target range of values for the biosignal from a set of workout effort ranges to the session.

9. A computing device comprising:
one or more memories; and
one or more processors in communication with the one or more memories and configured to execute instructions stored in the one or more memories to a method comprising:
determining a target time contribution for each of a set of workout target effort zones for a workout of a user, thereby determining target time contributions, wherein each of the set of workout target effort zones corresponds to a target range of values for a biosignal;
determining a time series of workout target effort zones for the user based on the target time contributions for the set of workout target effort zones;
receiving, during a workout time period, real-time biosignal data from a sensor in a wearable electronic device being worn by the user;
generating, during the workout time period, an audio, visual, or haptic stimulus based on the real-time biosignal data and a workout target effort zone in the time series of workout target effort zones; and
outputting, during the workout time period, the audio, visual, or haptic stimulus.

10. The computing device of claim 9, wherein the biosignal is a heart rate.

11. The computing device of claim 9, wherein each workout target effort zone in the time series of workout target effort zones includes a workout target effort zone from among the set of workout target effort zones, and wherein the audio, visual, or haptic stimulus identifies a particular workout target effort zone corresponding to a current time.

12. The computing device of claim 9, wherein the method further comprises:
estimating, based on the real-time biosignal data and at least part of the time series of workout target effort zones, an extent to which a current effort level of the user differs from the workout target effort zone; and
determining a stimulus property based on the extent, wherein the audio, visual, or haptic stimulus is generated based on the stimulus property.

13. The computing device of claim 12, wherein estimating, based on the real-time biosignal data and at least part of the time series of workout target effort zones, the extent to which the current effort level of the user differs from the workout target effort zone includes:
comparing a current value of the biosignal to the target range of values for the biosignal of the workout target effort zone.

14. The computing device of claim 9, wherein the audio, visual, or haptic stimulus includes an audio stimulus.

15. The computing device of claim 9, wherein determining the time series of workout target effort zones for the user based on the target time contributions for the set of workout target effort zones comprises using a model that transforms the biosignal to an estimated energy expenditure.

16. The computing device of claim 9, wherein determining the time series of workout target effort zones includes:
determining a target percentage of time to be spent across the set of workout target effort zones based on one or more preferences identified by the user;
defining a set of sessions for the workout, where each session corresponds to the time series of workout target effort zones; and
assigning, to each workout target effort zone of the time series of workout target effort zones, a duration of the workout time period and a particular target range of values for the biosignal from a set of workout effort ranges to the session.

17. A computer-readable medium storing a plurality of instructions that, when executed by one or more processors of a computing device, cause the one or more processors to perform a method comprising:
determining a target time contribution for each of a set of workout target effort zones for a workout of a user, thereby determining target time contributions, wherein each of the set of workout target effort zones corresponds to a target range of values for a biosignal;
determining a time series of workout target effort zones for the user based on the target time contributions for the set of workout target effort zones;
receiving, during a workout time period, real-time biosignal data from a sensor in a wearable electronic device being worn by the user;
generating, during the workout time period, an audio, visual, or haptic stimulus based on the real-time biosignal data and a workout target effort zone in the time series of workout target effort zones; and
outputting, during the workout time period, the audio, visual, or haptic stimulus.

18. The computer-readable medium of claim 17, wherein each workout target effort zone in the time series of workout target effort zones includes a workout target effort zone from among the set of workout target effort zones, and wherein the audio, visual, or haptic stimulus identifies a particular workout target effort zone corresponding to a current time.

19. The computer-readable medium of claim 17, further comprising:
estimating, based on the real-time biosignal data and at least part of the time series of workout target effort zones, an extent to which a current effort level of the user differs from the workout target effort zone; and
determining a stimulus property based on the extent, wherein the audio, visual, or haptic stimulus is generated based on the stimulus property.

20. The computer-readable medium of claim 17, wherein determining the time series of workout target effort zones includes:
determining a target percentage of time to be spent across the set of workout target effort zones based on one or more preferences identified by the user;
defining a set of sessions for the workout, where each session corresponds to the time series of workout target effort zones; and
assigning, to each workout target effort zone of the time series of workout target effort zones, a duration of the workout time period and a particular target range of values for the biosignal from a set of workout effort ranges to the session.
